(19) United States
(12) Patent Application Publication
(10) Pub. No.: US 2003/0125294 A1
(43) Pub. Date: Jul. 3, 2003

(54) Selective toxin expression in angiogenic endothelial cells

(75) Inventors: Ian H. Maxwell, Denver, CO (US); Francoise Maxwell, Denver, CO (US); Cortina Kaletta, Windach (DE); Kurt W. Naujoks, Penzberg (DE)

Correspondence Address: MORGAN LEWIS & BOCKIUS LLP, 1111 PENNSYLVANIA AVENUE NW, WASHINGTON, DC, 20004, US

(73) Assignee: University Technology Corporation

(21) Appl. No.: 10/294,903

(22) Filed: Nov. 15, 2002

Related U.S. Application Data
(63) Continuation of application No. 09/704,824, filed on Nov. 3, 2000, now abandoned.
(60) Provisional application No. 60/163,250, filed on Nov. 3, 1999.

(51) Int. Cl. A61K 48/00; C07H 21/04

(52) U.S. Cl. 514/44; 536/23.6; 536/23.7

(57) ABSTRACT

The present invention relates to pharmaceutical compositions capable of specifically inducing cell death in the proliferating angiogenic endothelial cells associated with solid tumors and other angiogenesis associated diseases. More specifically, the present invention relates to nucleotide constructs that are selectively active in angiogenic endothelial cells and that encode highly toxic agents, such as diphtheria toxin, for expression in such cells.

### FIELD OF THE INVENTION

The present invention relates to isolated nucleic acid sequences, associated expression vectors and delivery vehicles, and to the targeted delivery of nucleic acids and vectors that are capable of specifically inducing cell death in the proliferating angiogenic endothelial cells associated with tumors and metastases or other neovascular diseases such as diabetic retinopathy and similar ocular diseases, psoriasis and rheumatoid arthritis. More specifically, the present invention relates to nucleotide constructs that are selectively active in angiogenic endothelial cells and that encode highly toxic agents, such as diphtheria toxin, and express these agents in such cells.

### BACKGROUND

Treatment of cancer generally entails a combination of surgery, radiation therapy and/or chemotherapy. These treatment methods, while widespread, are far from completely effective. Surgery is limited by the ability of physicians to detect and remove suspect tumors, which is particularly difficult for advanced, metastatic cancers. Radiation and chemotherapy induce mild to severe side effects and may actually increase the risk of further tumor development. Further discussion of the problems of conventional cancer treatments are found in U.S. Pat. No. 5,837,283 to McDonald et al. (1998).

The advent of molecular biology has suggested a number of new techniques for the treatment of cancer. It is known, for example, that many tumors express a particularized set of genes and proteins compared with the genes and proteins expressed in normal tissue. This has lead to proposals to treat cancers with antibodies (immunotherapy) or with gene expression vehicles (gene therapy).

In immunotherapy, an antibody that is specific to an antigen uniquely or preferentially presented by a tumor is often used to direct a therapeutic or cytotoxic substance directly to the tumor cell. Although promising in theory, immunotherapy has not proved effective in treating solid tumors. Among the suspected reasons for this failure is the inability of antibodies to penetrate deep into solid tumors (Burrows et al. 1995 Clin. Cancer Res. 1(12): 1623-1634). Also, therapeutic antibodies can be destroyed by an anti-idiotype immune response. Therapeutic antibodies are also often limited to specific cancers, or even antigenic subpopulations of specific cancers.

Immunotherapy may also pose health risks to a patient. In particular, immunotherapy using ricin conjugates has been shown to cause vascular or capillary leak syndrome (De Vita et al. “Cancer, Principles and Practice of Oncology” (5th ed. 1997) Vol. 1, pp. 3045-3055). Vascular leak syndrome is characterized by decreased serum albumin levels, increased interstitial fluid levels and may cause pulmonary edema. Also, surface antigens, including for example the receptors for the vascular endothelial growth factor (VEGF) are present on non-proliferating endothelial cells, at least in relatively lower concentrations. Thus, antibodies directed to angiogenic endothelial cells might also kill non-angiogenic endothelial cells that surround healthy tissues.

Gene therapy procedures generally involve delivery of a gene expression vector that encodes a therapeutic or cytotoxic protein to a specific target cell, such as a tumor cell or tumor-associated angiogenic endothelial cell. For example, Huber et al. 1991 Proc. Nat'l Acad. Sci. USA 88(18): 8039-43 describes DNA vectors capable of directing expression, at least in cell culture, of the thymidine kinase (tk) gene in a tissue specific manner in “neoplastic” HepG2 cells. Expression of the tk gene renders these cells susceptible to treatment with the prodrug araM. AraM is not harmful to the cells, but it is converted by cell proteins in conjunction with thymidine kinase into the cytotoxic compound araATP.

Another example of such a gene therapy procedure is the expression of the DT-A chain coding sequence under the control of promoters and enhancers from immunoglobulin genes in B-lymphoid cells in cell culture. The plasmid was less active in expressing DT-A in a pre B-cell line than in B-lymphoma cells, thus allowing the sparing of normal B-cell progenitors. However the level of DT-A expression was unexpectedly low (Maxwell et al., 1991 Cancer Research 51:42099-4304).

As with immunotherapy, gene therapy is limited by the ability to deliver the gene expression vector to the entire tumor. Furthermore, to the extent that vector uptake cannot be limited solely to tumor cells, expression of the therapeutic or cytotoxic protein must be tightly regulated such that the protein is expressed only in the target cancer cells. This tight regulation of gene expression has the adverse effect of eliminating the general utility of any particular gene expression vector.

1. Therapies Based on the Transfection of Angiogenic Endothelial Cells

The difficulties with cancer therapies that target a tumor directly have led to proposals to treat tumors by inhibiting angiogenesis, i.e., by inhibiting the development of blood vessels that support the growing tumor (Burrows et al. 1995 Clin. Cancer Res. 1(12): 1623-1634). Inhibition of angiogenesis has several advantages over immunotherapy and attempts to transform tumor cells by gene therapy. Unlike the solid tumor mass, the endothelial cells of growing capillaries are relatively more accessible to a variety of therapeutic agents. Furthermore, because angiogenesis is a requirement common to essentially all solid tumors, methods of inhibiting angiogenesis, at least in theory, would be likely to be broadly applicable to solid tumors of any lineage.

For example, the McDonald et al. U.S. Pat. No. 5,837,283 describes the selective targeting of angiogenic endothelial cells with lipid/DNA complexes, including cationic liposomes, to deliver a substance that affects the targeted cells by inhibiting or promoting their growth. As an example of such substances, this patent describes (col. 12, lines 55-56) the use of nucleotide sequences that encode a protein that kills angiogenic endothelial cells. Preferred sequences are linked to a promoter that is selectively activated only within the environment of an angiogenic endothelial cell. Specific constructs suggested by McDonald et al. include the coding sequence for the Herpes simplex thymidine kinase gene (with patient therapy involving the subsequent administration of gancyclovir). Antisense molecules that block expression of proteins necessary for the cell's survival are more generally suggested.

The Mueller et al. U.S. Pat. No. 5,885,833 (1999) discusses the use of nucleic acid constructs for gene therapy purposes that comprise an activator sequence, a promoter module, and a structural gene. A specific construct was described as including a promoter module that further comprises a CHR region and a nucleotide sequence that binds a protein of the E2F family. This Patent describes (for example at col. 8, lines 5-9) the targeting and transformation of endothelial cells to express a protein that directly or indirectly inhibits the proliferation of, or kills proliferating endothelial cells. The use of DNA encoding several such “anti-inflammatory” or “proliferation inhibitor” proteins are suggested, for example, at col. 9, line 45 to col. 10, line 50. These include the retinoblastoma protein (pRb/p110 or its analogues p107 and p130), the p53 protein, the p21 (WAF-1) protein, the p16 protein, other cdk inhibitors, the GADD45 protein and the bak protein. See U.S. Pat. No. 5,885,833, col. 10, lines 1-19.

Notably, the proteins of Mueller et al. are cell-cycle regulators that affect growth negatively rather than toxins. Moreover, the recited proteins are part of complex regulatory systems, and do not directly produce apoptosis (i.e., cell death). For example, p53 is a DNA-binding protein that can regulate transcription. Higher levels of p53 cause an arrest of progression through the cell cycle, but not cell death. (Prives et al. 1993 Curr. Opin. Cell Biol. 5:214-8). Similarly, P16 and other cdk inhibitors affect cyclin-dependent kinases (CDKs) that phosphorylate substrates to promote an orderly progression through the cell cycle. Notably, these proteins are ubiquitous in human cells and therefore not toxic per se. Indeed, Haas-Kogan et al., 1995 EMBO J 14:461-472, describes the inhibition of apoptosis by the retinoblastoma gene product in a human osteosarcoma cell line.

Similarly, Sedlacek et al. U.S. Pat. No. 5,830,880 (1998) describes the genetic therapy of tumors with gene expression vectors containing a cell-cycle dependent promoter and endothelium specific activation sequences upstream of a DNA sequence encoding an antitumor substance. While this Patent suggests the use of cytotoxic agents to kill tumor cells, it also refers to the inhibition of the proliferation of endothelial cells at col. 6, lines 43-45. Antitumor substances described in this Patent include cytotoxic or cytostatic substances directed at a tumor, such as the interferons; angiogenesis inhibitors, such as angiostatin; and anti-proliferative proteins, such as p53 or the retinoblastoma protein. Specific examples of DNAs encoding such anti-proliferation substances are provided at col. 6, lines 48-64, and also include the retinoblastoma protein (pRb/p110 or its analogues p107 and p130), the p53 protein, the p21 (WAF-1) protein, the p16 protein, other cdk inhibitors, the GADD45 protein and the bak protein. Such constructs may inhibit endothelial cells, but are not toxic (or highly toxic) per se.

Williams et al. U.S. Pat. No. 5,916,763 (1999) discloses nucleic acid molecules and associated vectors comprising a VEGF receptor promoter region, and in particular, the Flt-1 promoter. The Patent notes that tissue-specific expression of heterologous genes or DNA in endothelial cells would be desirable, because conventional drug delivery technology is not amenable to the tissue specific delivery of bioactive species. Accordingly, the Williams et al. specification describes methods of directly delivering bioactive species to endothelial cells, including vascular endothelial cells, via their specific endogenous production in the endothelium and describes vectors that are said to be useful for treating various diseases affecting or associated with the vascular endothelium. The Patent lacks, however, disclosure of an actual construct that directs expression of a highly toxic protein. Also lacking is any disclosure or showing suggesting how such a construct might be formulated or its actual effect on angiogenic cells or cell lines.

2. Limited Therapeutic Use of Toxins Targeted to Endothelial and Other Cells

It has been suggested that various protein toxins could be encapsulated and targeted for delivery, for example, to tumor associated capillary endothelial cells, as described in Hawrot et al. U.S. Pat. No. 4,948,590 (1990). This Patent is generally directed to avidin or streptavidin conjugated liposomes containing, for example, ricin A or diphtheria toxin. Other examples of toxins directed to endothelial cells include Thorpe et al. U.S. Pat. No. 5,965,132 (1999), which discusses the use of antibody conjugates directed to the vasculature of solid tumors. In particular, Thorpe et al. describes an antibody conjugate that binds to a complex of growth factor and growth factor receptor present on the surface of intra tumoral blood vessels of the vascularized tumor, but that does not bind to the individual growth factor or growth factor receptor. Thorpe et al. also describes antibody conjugates in which the relevant antibody is linked to various cytotoxins, such as deglycosylated ricin A or diphtheria toxin, or diagnostic agents. Similarly, diphtheria toxin has been suggested for use as a cytotoxic agent as a component of an immunotoxic construct targeted to the FLK-1 receptor for VEGF on endothelial cells. See, for example, Ullrich et al. U.S. Pat. No. 5,851,999 (1998).

The Williams et al. Patent suggests that VEGF regulatory sequences can be used to target endothelial cells for killing. The Patent indicates that tumor cells or infected cells can be targeted for death, as has been done using antibodies specific for the tumor cell or the infected cell to deliver a toxic agent to the diseased cell. However, the Williams et al. Patent does not provide details about the use of such toxic agents, and essentially only mentions that the disclosed regulatory sequences can be used to target expression of a “toxic” peptide of bacterial, plant or animal origin to an endothelial cell, preferably in the vascular endothelium. Any of a number of toxic proteins of bacterial origin, such as Pseudomonas exotoxin A or Diphtheria toxin, and animal-derived toxic proteins such as tumor necrosis factor-alpha, are said to be appropriate for this purpose. The Patent indicates that such toxic proteins also may act as antiviral agents or an antitumor agents.

In general, where specific constructs appear to have been developed and tested, the use of nucleotide constructs to transfect target cells to express toxins such as ricin and diphtheria toxin does not appear to be favored in the medical literature. The Chiocca et al. U.S. Pat. No. 5,688,773 (1997) explains that once released locally, for example in the nervous system, toxins could cause toxicity in a variety of non-targeted tissues including blood vessels and bone marrow and that transformed cells could not be selectively controlled. This risk of toxicity may be exacerbated if such toxins were to be released systemically into the circulatory system.

Thus, it may be noteworthy that the McDonald et al., Mueller et al. and Sedlacek et al. patents described above with regard to the genetic therapy of angiogenic endothelial cells generally suggest the use of a wide variety of agents that inhibit cellular proliferation or enhance localized inflammation-mediated immune response. However, these patents do not teach the use of constructs encoding highly toxic proteins, such as diphtheria toxin. Until the present invention, it had not been known that diphtheria toxin, for example, could be safely and selectively expressed in angiogenic endothelial cells and demonstrate a therapeutic anti-tumor activity.

In non-endothelial cell systems, Murayama et al. described the development of a cell-specific expression system to administer a DT-A chain “suicide gene” selectively in tumor cells by constructing a plasmid containing the diphtheria toxin A (DT-A) fragment linked to human alpha-fetoprotein (AFP) promoter and enhancer, and tested whether it could exert its cytocidal effect selectively on AFP-producing cells (J Surg Oncol 1999 Mar; 70(3):145-9). Their results indicated that selective killing of AFP-producing cells will be attained by introducing the DT-A gene linked to the promoter and enhancer region of AFP.

Similarly, Tana et al. reported the antitumor effect of diphtheria toxin A-chain gene-containing cationic liposomes conjugated with monoclonal antibody directed to tumor-associated antigen of bovine leukemia cells, in Jpn J Cancer Res 1998 Nov; 89(11):1202-11. These investigators administered the monoclonal antibody c143 against tumor-associated antigen (TAA) expressed on bovine leukemia cells in a form conjugated to cationic liposomes carrying a plasmid pLTR-DT which contained a gene for diphtheria toxin A-chain (DT-A) under the control of the long terminal repeat (LTR) of bovine leukemia virus (BLV) in the multicloning site of pUC-18. Injections of pLTR-DT-containing cationic liposomes coupled with c143 into tumor-bearing nude mice resulted in significant inhibition of tumor growth.

3. Regulation of Transcription in Angiogenic Endothelial Cells

Several transcription control sequences, such as promoters, enhancers and transcription factor binding sites, that are specifically or preferentially active in proliferating endothelial cells have been characterized. A variety of transcription control sequences, specifically promoters that are activated in endothelial or angiogenic endothelial cells are described in the McDonald et al., Mueller et al., and Sedlacek et al. patents discussed above. The disclosures of these patents (and all other documents identified in this specification) are specifically incorporated by reference in their entirety.

Examples of such transcription control sequences include sequences derived from the genes for endoglin, the VEGF receptors (flt-1 and flk-1), von Willebrand factor, tie, tie2, ets-1, endothelin, endosialin, E-selectin, VE-cadherin. (See, for example, the Sedlacek et al. Patent noted above.) Transcription control sequences also include individual elements or larger sequences such as promoters. These endothelial cell-specific elements include the binding sites for transcription factors ETS-1 and GATA-2, which have consensus binding sites 5′-GGA(A/T)-3′ and 5′-TTATCT-3′, respectively. Use of such control sequences may specifically or preferentially constrain expression of a desired gene product to the angiogenic endothelium.

Recently, several highly specific enhancers have been identified. These are DNA sequences that bind certain proteins (e.g., transcription factors) that only are found in certain types of cells and which modulate the transcriptional activity of cis-linked DNA-sequences. These enhancer binding proteins are activators of transcription which regulate the expression of certain genes that are therefore expressed only in these cells or which become transcriptionally active only under certain conditions. For example, such an autonomous endothelial cell-specific enhancer is found in the first intron of the mouse tie2 gene, as described by Schlaeger et al. 1997 Proc. Natl. Acad. Sci. USA. 94: 3058-3063. The corresponding enhancer from the first intron of VEGF-Receptor-2 (FLK-1) is described by Kappel et al. 1999 Blood 93:4284-92, and from the first intron of the ets-1 gene by Jorcyk et al., 1997 Cell. Mol. Biol. 43:211-225. Another vascular endothelial cell-specific enhancer (HB-EGF enhancer) is described in Lee et al. U.S. Pat. No. 5,656,454 (1997) for the purpose of treating arteriosclerosis.

Other regulatory elements are responsive to hypoxic conditions, and hypoxia has been shown to be a very important stimulus for new vessel formation, as seen with respect to coronary artery disease, tumor angiogenesis and diabetic neovascularization. Low oxygen conditions also may be induced by a drug delivered to a tumor. Webster et al. U.S. Pat. No. 5,834,306 (1998) relates to specific hypoxia response enhancer elements from the group of erythropoietin HRE, pyruvate kinase HRE, enolase 3 HRE and endothelin-1 HRE elements. The expression of a selected gene was shown to be enhanced in the target tissue under hypoxic conditions. Liu et al. 1995 Circ. Res. 77:638-643 describe a 5′ element to the HIF (hypoxia-inducible factor-1) consensus sequence that can act as a hypoxia-specific enhancer when placed upstream or downstream from a heterologous promoter. Two or more copies of the enhancer may be arranged in tandem to increase expression of the heterologous protein to even higher levels.

4. Delivery of Nucleotide Constructs Targeted to Angiogenic Endothelial Cells

Selective gene expression in angiogenic endothelial cells may also be facilitated by the method of delivery. For example, certain cationic liposomes or polynucleotide/lipid complexes are preferentially taken up by angiogenic (proliferating) endothelial cells. See, for example, the McDonald et al. Patent discussed above and the various constructs described therein. Nucleic acid delivery systems or agents other than liposomes are also known in the art and are contemplated to be of use in the invention described herein. See, e.g., Wu et al. U.S. Pat. No. 5,166,320 (1992), U.S. Pat. No. 5,635,383 (1997) and U.S. Pat. No. 5,874,297 (1999); Hartmut U.S. Pat. No. 5,354,844 (1994) and U.S. Pat. No. 5,792,645 (1998); and Gopal U.S. Pat. No. 5,670,347 (1997).

Retroviral vectors are also of interest because of the requirement of cell division for retroviral integration, which would favor uptake by proliferating cells, such as angiogenic epithelial cells, over non-proliferating cells. However, the specific method of delivery according to the present invention is of relatively less concern when constructs are utilized that are preferentially transcribed in angiogenic endothelial cells.

Accordingly, as indicated above, there is a need in the medical field for expression vectors, and methods of expression vector delivery, capable of specifically and effectively inducing cell death in the proliferating angiogenic endothelial cells associated with solid tumors.

### SUMMARY OF THE INVENTION

It is an object of the present invention to provide nucleotide constructs that are selectively active in angiogenic endothelial cells and that encode highly toxic agents for expression in such cells. Contemplated agents include certain bacterial and other toxins, such as diphtheria toxin, pseudomonas exotoxin, cholera toxin, shiga-like toxin I, ricin A, trichoanguin, alpha-trichosanthin, abrin A, modeccin, Granulysin and related toxins, and highly toxic mutants and fragments of the foregoing. Especially preferred are diptheria toxin and diptheria toxin mutants and fragments thereof. It is a further object of the present invention to provide nucleotide constructs for the controlled activation of such agents, and to provide constructs and delivery methodologies that do not result in systemic cell death.

In a preferred embodiment, this invention relates to an expression vector comprising an endothelial cell specific promoter, which is inducible in proliferating endothelial cells, preferably the E-selectin and the ets-1 promoters and a DNA-sequence that encodes a bacterial toxin like the DT-A-chain of Diphtheria toxin and mutants of such toxins (for example, tox176). Other promoters contemplated by the present invention include but are not limited to endosialin promoter, flt-1 promoter, flk-1 promoter and KDR promoter. The present invention also includes an expression vector further comprising an enhancer, in one or more copies, that is active in endothelial cells and angiogenic endothelial cells. Enhancers that are contemplated by the present invention include, but are not limited to, HB-EGF enhancer, enhancer from the first intron of the mouse tie2 gene, enhancer from the first intron of the gene encoding VEGF receptor (flk-1/KDR), enhancer from the first intron of the ets-1 gene, and a hypoxic response enhancer element that is selectively active under hypoxic conditions. Preferred hypoxic response enhancer element includes but are not limited to erythropoietin HRE element, pyruvate kinase HRE element, enolase HRE element, endothelin-1 HRE element, and VEGF hypoxic regulated enhancer.

It is contemplated that any means available in the art for the transfer of such nucleotide constructs into animals including humans, can be utilized. Such means include viral vectors, particularly retroviral and parvoviral vectors, as well as other methods like liposomes, preferably cationic liposomes, liposomes and cationic liposomes attached to agents such as antibodies and other proteins that target the liposomes selectively to endothelial and angiogenic endothelial cells, and polynucleotide lipid complexes.

The present invention also relates to pharmaceutical compositions comprising an isolated nucleic acid comprising a sequence encoding a highly toxic protein and regulatory elements effective to selectively express the sequence in angiogenic endothelial cells and delivery vehicles. Examples of delivery vehicles include but are not limited to retroviral particles, parvoviral particles, liposomes, cationic liposomes, liposomes or cationic liposomes with attached agents that target the liposomes selectively to endothelial and angiogenic endothelial cells respectively, and polynucleotide lipid complexes.

The present invention also relates to the use of the pharmaceutical compositions to treat angiogenesis associated diseases and diseases involving pathological blood vessel proliferation such as rheumatoid arthritis, atherosclerosis, diabetes mellitus, retinopathy, psoriasis and retrolental fibroplasia.

It is also contemplated that the present invention may be used in conjunction with other anti-tumor or anti-angiogenic therapeutics or therapies. Anti-tumor therapies include known chemotherapeutics, such as cisplatin, and radiation therapy. Anti-angiogenic therapies include administration of compounds such as angiostatin, thalidomide, and anti-VEGF antibodies.

### BRIEF DESCRIPTION OF THE DRAWINGS

FIG. 1. FIG. 1 shows the relative effects of DT-A and DT-A-tox176 expression on protein synthesis in HUVEC as determined by the decrease in luciferase activity relative to non-DT-A transfected cells.

FIG. 2. FIG. 2 shows luciferase activity in HUVEC transfected with an E-selectin promoter/luciferase expression vector (pE-S-GFP-LUC) alone, or in the presence of TNFα or lipopolysaccharide (“LPS”).

FIG. 3. FIG. 3 shows the effect of transfecting increasing amounts of DT-A expression vector (pE-S-DT-A) into HUVEC cells cotransfected with an E-selectin/luciferase expression vector. Cells were cultured in the presence or absence of TNFα and assayed 9.5 or 19.5 hours post transfection.

FIG. 4. FIG. 4 shows luciferase expression in 324K cells electoporated with etbz-EX-GFP-Luc or pE-S-GFP-Luc and pEVRFETS(ets) expression plasmid.

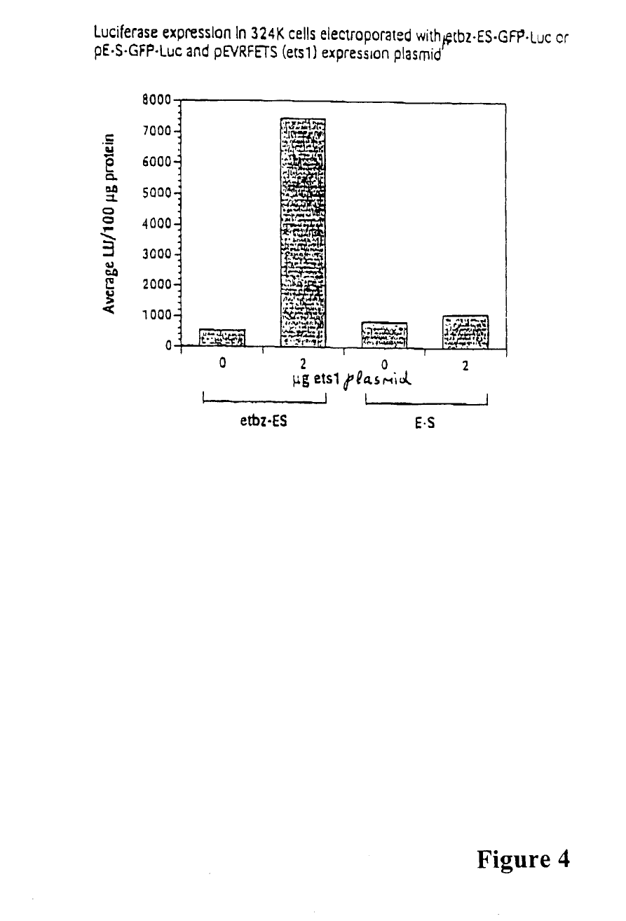

### DETAILED DESCRIPTION OF THE INVENTION

The present invention is based on the discovery that pharmaceutical compositions comprising nucleotide constructs that are provided in certain delivery vehicles are capable of specifically inducing cell death in the proliferating angiogenic endothelial cells associated with solid tumors and other angiogenesis associated diseases. More specifically, the present invention is based on the discovery that particular nucleotide constructs which encode highly toxic agents are selectively active in angiogenic endothelial cells.

1. Definitions.

Unless defined otherwise, all technical and scientific terms used in this specification shall have the same meaning as commonly understood by persons of ordinary skill in the art to which the present invention pertains.

“Angiogenesis” refers to the formation of new blood vessels. Endothelial cells form new capillaries in vivo when induced to do so, such as during wound repair or in tumor formation or certain other pathological conditions.

The term “angiogenesis-associated disease” refers to certain pathological processes in humans where angiogenesis is abnormally prolonged or pathologically induced. Such angiogenesis-associated diseases include diabetic retinopathy, chronic inflammatory diseases, rheumatoid arthritis, dermatitis, psoriasis, stomach ulcers, and most types of human solid tumors.

“Angiogenic endothelial cells” refers to endothelial cells undergoing angiogenesis which are proliferating at a rate substantially higher than the normal proliferation rate for endothelial cells in general.

“Combination” or “co-administration” refers to an administration schedule that is synchronous, serial, overlapping, alternating, parallel, or any other treatment schedule in which the various agents or therapies are administered as part of a single treatment regimen, prescription or indication or in which the time periods during which the various agents or therapies that are administered otherwise partially or completely coincide.

“Delivery vehicle” refers to a vector or other pharmaceutically acceptable component(s) that contain or are associated with the nucleic acid constructs according to the present invention. Such delivery vehicles include those known in the art,-including retroviral particles, parvoviral particles, liposomes, including cationic liposomes, and polynucleotide lipid complexes.

“Endothelial cells” refers to those cells making up the endothelium, which is the monolayer of cells that line the inner surface of the blood vessels of the circulatory system. These cells retain a capacity for cell division, although they proliferate very slowly under normal (that is, non-angiogenic) conditions, undergoing cell division only about once a year.

“Enhancer” refers to a DNA-sequence that modulates the transcriptional activity of cis linked sequences. An enhancer may be located upstream, downstream or embedded within the coding region of the DNA sequence. One or more copies of the enhancer may be used to increase expression of a heterologous protein, including the increase of expression in a tissue specific manner.

“Heterologous” refers to a nucleotide sequence, such as a promoter, or to a protein-encoding sequence, that does not occur naturally as part of the DNA sequence in which it is present.

“Highly toxic” or “highly toxic agent” refers to a protein or peptide that is expressed in a target cell and inhibits the synthesis of protein, DNA or RNA, or destabilizes the lipid surface, or otherwise results in cell death by apoptosis or necrosis.

“Nucleotide construct” refers to a DNA or other nucleic acid molecule that has been subjected to molecular manipulation in vitro.

“Promoter” refers to a DNA sequence that functions to control the transcription of one or more genes, located upstream with respect to the direction of the transcription initiation site of the gene. It contains a binding site for DNA-dependent RNA polymerase, transcription initiation sites and, in some case, other DNA sequences, including, but not limited to transcription factor binding sites, repressors and activator binding sites, calcium, serum or cAMP responsive elements, and any other sequences of nucleotides known to one of skill in the art to act directly or indirectly to regulate the amount of transcription from the promoter.

“Selectively active” refers to a level of transcription or expression of a particular nucleotide construct in a particular type, category or group of cells, that is significantly higher than the level of transcription or expression of that construct in cells of other types, categories or groups, preferably where such other types, categories or groups of cells have a substantially zero or otherwise relatively low basal level of transcription or expression for that construct. For example, the constructs of the present invention are selectively active in endothelial cells relative to non-endothelial cells, and, preferably are selectively active in angiogenic endothelial cells relative to quiescent or non-angiogenic endothelial cells.

“Therapeutically effective” refers to an agent that is effective to reduce the amount or the rate of the process of angiogenesis or neovascularization, preferably to substantially prevent the continuation of such processes at existing sites of angiogenesis, or to substantially prevent the initiation of angiogenesis at additional, undesirable sites of angiogenesis. For example, in the case of treating angiogenesis related to tumor metastasis, a therapeutically active or effective agent would show significant antitumor activity or tumor regression through inhibition of angiogenesis. Such a compound might, for example, reduce primary tumor growth and, preferably, the metastatic potential of a cancer. Alternatively, such a compound might reduce tumor vascularity, for example either by decreasing microvessel size or number or by decreasing the blood vessel density ratio.

“Tumor regression” refers to a decrease in the overall size, diameter, cross section, mass ir viability of a tumor; tumor marker reduction or a positive indication from other conventional indicia of cancer diagnosis and prognosis that indicates a reduction or growth slowing of cancer cells, as a result of the treatment of a cancer patient with compositions according to the present invention. Preferably, the administration of such compounds results in at least about a 30 percent to 50 percent tumor regression, more preferably at least about a 60 to 75 percent tumor regression, even more preferably at least about an 80 to 90 percent tumor regression and most preferably at least about a 95 or a 99 percent tumor regression at one or more tumor sites in a cancer patient. Ideally, such administration results in the killing or eradication of viable tumor cells or completely eradicates the tumor cells at one or more tumor sites in a cancer patient, leading to a clinically observable remission or other enhancement in health of a patient.

2. Detailed Description

A. Diphtheria Toxin (DT) and Regulated Expression of the DT-A Chain

Diphtheria toxin (DT) of Corynebacterium diphtheriae is translated as a single polypeptide chain which is post-translationally cleaved into the A (Mr 21,167) and B (Mr 37,195) fragments, and these remain linked via a disulfide bond (Pappenheimer, (1977). Ann. Rev. Biochem. 46, 69-94). The B-chain of DT facilitates entry of the A-chain into the cytoplasm by binding to a cell surface transmembrane protein (related to the epidermal growth factor precursor, Naglich, et al.(1992). Cell 69, 1051-1061), followed by receptor mediated endocytosis into endosomes. Upon acidification of the endosome the disulfide bond is reduced and the A-chain is delivered into the cytoplasm (O'Keefe, et al. (1992) Proc. Natl. Acad. Sci. USA 89, 6202-6206). A mechanism by which the B-chain participates in this delivery has been suggested from the structure of DT, determined by X-ray crystallography (Choe, et al. (1992). Nature (London) 357, 216-222). The diphtheria toxin A-chain is an ADP-ribosyltransferase specific for a post-translationally modified histidine residue, diphthamide, of eukaryotic elongation factor-2 (eEF-2), its only known substrate. Transfer of an ADP-ribose moiety from NAD+ to eEF-2 inhibits protein synthesis and leads to cell death (Collier, (1990). ADP-Ribosylating Toxins and G Proteins: Insights into Signal Transduction, J. Moss and M. Vaughan, Eds. (American Society of Microbiologists, Washington, D.C.) pp. 3-19) either by induction of an apoptosis program or by other mechanisms (Keppler-Hafkemeyer, et al. (1998). Biochemistry 37, 16934-16942). Due to its enzymatic activity, DT-A is extremely toxic and a single molecule may be sufficient to cause cell death (Yamaizumi, et al. (1978). Cell 15, 245-250).

The regulated expression of a toxin gene can be used as an efficient means of killing specific cell types both in vitro and in transgenic animals (Maxwell, et al. (1986) Cancer Res. 46, 4660-4664; Palmiter, et al. (1987) Cell 50, 435-443; Breitman, et al. (1987) Science 238, 1563-1565). We have previously proposed such a use of the diphtheria toxin A-chain (DT-A) gene as a therapeutic agent for cancer (Maxwell, et al. (1986) Cancer Res. 46, 4660-4664). Successful therapy would depend both on efficient delivery and appropriately targeted expression of the DT-A gene. Since DT-A may be lethal when introduced into cells at a level as low as one molecule per cell (Yamaizumi, et al. (1978). Cell 15, 245-250), in theory only minimal expression of the DT-A gene might suffice to ablate targeted cells. Any release of DT-A from lysed cells also might not be harmful to adjacent tissues, because DT-A is unable to enter cells in the absence of the B-chain.

The DT-A gene, therefore, potentially offers a highly specific therapeutic agent, provided sufficiently stringent regulation of its expression can be imposed. The feasibility of this requirement in vivo has been demonstrated in transgenic mice. Thus, by using tissue-specific transcriptional regulatory elements, it has been possible to target the expression of DT-A with sufficient stringency to cause ablation of the targeted tissue, e.g., exocrine pancreas (Palmiter, et al. (1987) Cell 50, 435-443) or ocular lens (Breitman, et al. (1987) Science 238, 1563-1565; Kaur, et al. (1989) Development, 105, 613-619)) without damage to other tissues. Such mice are able to breed and can transmit the ablated phenotype (Breitman, et al. (1987) Science 238, 1563-1565; Behringer, et al. (1988) Genes & Development 2, 453-461; Kaur, et al. (1989) Development, 105, 613-619.). This approach might be applied in appropriate systems using regulatory elements that display a low level of leaky expression in non-target tissues by employing an attenuated mutant of DT-A known as tox176 (Maxwell, et al. (1987). Mol. Cell. Biol. 7, 1576-1579).

B. Preparation of Nucleotide Constructs

The present invention further provides recombinant nucleotide constructs that contain a coding sequence. Methods for generating nucleotide constructs are well known in the art, for example, see Sambrook et al., Molecular Cloning (1989). In the nucleotide constructs, a coding DNA sequence is operably linked to expression control sequences and/or vector sequences.

The choice of vector and/or expression control sequences to which one of the toxin encoding sequences of the present invention is operably linked depends directly, as is well known in the art, on the functional properties desired, e.g., protein expression, and the host or target cell to be transformed. A nucleotide construct contemplated by the present invention is at least capable of selectively expressing the structural toxin gene operably linked to the vector and/or expression control sequences and, preferably also capable of directing replication or inserting into the genome of eukaryotic or prokaryotic host or target cell as required.

Expression control elements that are used for regulating the expression of an operably linked protein encoding sequence are known in the art and include, but are not limited to, inducible promoters, constitutive promoters, enhancers, secretion signals, and other regulatory elements. Expression vectors in which expression is restricted solely to angiogenic endothelia are most preferred, although selective expression in angiogenic endothelial cells is contemplated. Also preferred are the inducible promoters that are readily controlled, such as being responsive to a nutrient in the target or host cell's medium or environment.

Nucleotide constructs or expression vectors compatible with eukaryotic cells, preferably those compatible with vertebrate cells, can also be used to form nucleotide constructs that contain a coding sequence. Eukaryotic cell expression vectors are well known in the art and are available from several commercial sources. Typically, such vectors are provided containing convenient restriction sites for insertion of the desired DNA segment. Typical of such vectors are pSVL and pKSV-10 (Pharmacia), pBPV-1/pML2d (International Biotechnologies, Inc.), pTDT1 (ATCC, #31255), the vector pCDM8 described herein, and the like eukaryotic expression vectors.

Eukaryotic cell expression vectors used to construct the nucleotide constructs of the present invention may further include a selectable marker that is effective in an eukaryotic cell, preferably a drug resistance selection marker. A preferred drug resistance marker is the gene whose expression results in neomycin resistance, i.e., the neomycin phosphotransferase (neo) gene. (Southern et al., J. Mol. Anal. Genet 1:327-341, 1982.) Alternatively, the selectable marker can be present on a separate plasmid, and the two vectors are introduced by co-transfection of the host cell, and selected by culturing in the appropriate drug for the selectable marker.

In one embodiment, the vector containing a coding nucleic acid molecule will include a prokaryotic replicon, i.e., a DNA sequence having the ability to direct autonomous replication and maintenance of the recombinant DNA molecule extrachromosomally in a prokaryotic host cell, such as a bacterial host cell, transformed therewith. Such replicons are well known in the art. In addition, vectors that include a prokaryotic replicon may also include a gene whose expression confers a detectable marker such as a drug resistance. Typical bacterial drug resistance genes are those that confer resistance to ampicillin or tetracycline.

Vectors that include a prokaryotic replicon can further include a prokaryotic or bacteriophage promoter capable of directing the expression (transcription and translation) of the coding gene sequences in a bacterial host cell, such as E. coli. A promoter is an expression control element formed by a DNA sequence that permits binding of RNA polymerase and transcription to occur. Promoter sequences compatible with bacterial hosts are typically provided in plasmid vectors containing convenient restriction sites for insertion of a DNA segment of the present invention. Typical of such vector plasmids are pUC8, pUC9, pUC18, pUC19, pBR322 and pBR329 available from BioRad Laboratories, (Richmond, Calif.), pPL and pKK223 available from Pharmacia, Piscataway, N.J.

C. Transcription Regulatory Elements

As described above, nucleotide constructs of the present invention comprising transcription regulatory elements, such as promoters and/or enhancers are contemplated. The function of promoters and enhancers are well known to the art. Promoters direct the initiation of RNA transcription at discrete locations relative to the position of the promoter itself. Characteristic sequences found in eukaryotic promoters are the “CAAT” and “TATA” boxes. Promoters may also confer tissue specific, developmentally regulated or inducible expression patterns on the expressed sequences depending on the presence and arrangement of various cis acting sequences in the promoter. Enhancers are DNA sequences that often increase the transcription level from a given promoter. Like promoters, the effect of an enhancer on transcription is often tissue specific, developmentally regulated or inducible depending on the presence and arrangement of various cis acting sequences present. One notable feature of enhancers is that their effect on transcription can be independent of their position and orientation relative to a given promoter.

Nucleotide constructs of the invention in which a toxin gene is selectively expressed in angiogenic epithelia are contemplated. Consequently, epithelial specific control sequences are of interest, several of which have been described. For example, WO Patent 97/17359 describes constructs which contain the flt-l promoter operatively linked to genes encoding various gene product. Another patent (WO 97/00957) discloses the KDR/flk-1 promoter directed endothelial cell-specific transcription of a polypeptide-encoding sequence or an antisense template to which it is operably linked. Yet another patent describes the TIE-Promoter (U.S. Pat. No. 5,877,020). U.S. Pat. No. 5,747,340 describes a vector for expression of a nucleic acid cassette in bronchial epithelial and vascular endothelial cells comprising a segment of the 5′-flanking region of the preproendothelin-1 gene, and a nucleic acid cassette for expression.

Other promoters of interest for use in constructs according to the present invention include the endosialin gene, the endoglin gene, the E-selectin gene and the ets-1 gene. Endosialin is a cell surface glycoprotein of vascular endothelial cells in human cancer (Rettig W. et. al 1992 Proc. Natl. Acad. Sci. USA 89:10832-10836). The promoter region of the endoglin gene was described by Rius et al., in Blood 1998; 92:4677-4690. E-selectin expression is very low in normal adult blood vessels, but is significantly elevated in newly produced tumor capillaries. See, for example, Walton et al. (Anticancer Research, 1989; 18:1357-1360) which shows that the expression of a reporter gene from the E-selectin promoter in transduced endothelial cells generates a 30-fold increase of the gene compared to untreated cells by TNF-α. ETS-1, a transcription factor, is positively auto-regulated by its own gene product and the gene structure has been described (Jorcyk C. L. et al. 1991). The ETS transcription factor has a DNA-binding domain that binds a core GGA(A/T) DNA sequences. ETs binding sites are essential for promoter activity of many genes expressed in endothelial cells and involved in angiogenesis.

Other genes expressed during angiogenesis and contemplated as providing useful transcription control sequences, include VE-cadherin (Gory et al., 1998; 273; 6750-5), von Willebrand factor (Schwachtgen et al., 1997; Oncogene 15:3091-102), P and E-selectin, Endoglin (Rius et al., 1998; Blood 12:4677-4690), flt-1 (Morishita et al., 1995; J. Biol. Chem. 270:27948-27953).

D. Delivery of Constructs and Vectors to Endothelial Cells

Methods of delivery of nucleic acids to eukaryotic cells and related delivery vehicles are well known in the art. See Ausubel et al. (eds.) Current Protocols in Molecular Biology (1999) Ch. 9. Nucleic acid constructs and vectors of the invention may be delivered to endothelial cells by any available means, including but not limited to, transfection facilitating proteins and lipids, viral delivery vectors, “gene guns” as well as naked nucleic acid. See Teifel et al. Endothelium 5:21-35 (91997) and U.S. Pat. No. 5,837,283. For instance, nucleic acid constructs and vectors may be delivered to angiogenic endothelial cells by introduction into the circulatory system or by direct injection into the tumor mass. See U.S. Pat. No. 5,837,283.

Liposomal delivery is a preferred method of delivery. The term “liposome” is intended to be interpreted broadly and to encompass microparticulate colloidal systems, especially lipid or lipophilic vesicles, that are capable of encapsulating a drug or nucleotide construct. See Meyers (ed.) Molecular Biology and Biotechnology (1995) 260, 514. Liposome formulation and use is well known in the art. For example, Scheule et al. U.S. Pat. No. 5,948,767 describes cationic lipophilic amphiphiles suitable for delivery of therapeutically effective amounts of biologically active molecules to patients. Any liposomal formulation suitable for intravenous or local or direct administration to a tumor ass is to be preferred. Especially preferred are those liposome formulations that are referential taken up by angiogenic epithelial cells as described in U.S. Pat. No. 5,837,283 to McDonald et al. Also preferred are liposomes and cationic liposomes with attached agents, such as antibodies and other proteins, that target the liposomes selectively to endothelial and angiogenic endothelial cells. Other delivery methods of interest to the practitioner which involve nucleic acid complexes are described in U.S. Pat. No. 5,166,320 (1992), U.S. Pat. No. 5,635,383 (1997) and U.S. Pat. No. 5,874,297 (1999) to Wu et al., U.S. Pat. No. 5,354,844 (1994) and U.S. Pat. No. 5,792,645 (1998) to Hartmut and U.S. Pat. No. 5,670,347 (1997) to Gopal.

Retroviral expression vectors are another contemplated delivery vehicle of the invention. The construction of retroviral vectors is well known in the art. See Ausubel et al. (eds.) Current Protocols in Molecular Biology (1999) Ch. 9. Retroviral vectors require cell division for integration of the vector into the host or target cell genome. This characteristic renders such vectors of questionable use for diseases in which cell division is not present, such as cystic fibrosis. See Welsh et al. U.S. Pat. No. 5,958,893. However, the retroviral nucleotide constructs of the invention are preferably directed to angiogenic or proliferating epithelia. A benefit of the retroviral vectors contemplated by the invention is that they are not taken up by the quiescent or non-proliferating epithelia associated with healthy tissues. One concern regarding the use of retroviral expression vectors is limiting the risk that such vectors might revert to wild-type virus in the presence of residual helper viruses or transfer packaging capabilities to their target cell. Danos et al. U.S. Pat. No. 5,955,331 describes the generation of helper-free recombinant retroviruses that cannot transfer or acquire packaging functions to their target cells.

Recombinant parvoviral expression vectors are also contemplated by the present invention. Recombinant parvoviral vectors have been engineered for tumor specific expression of interleukin genes (Russell et al., J. Virol. 66: 2821-2828). Parvoviral vectors have also been engineered to produce parvoviral antigens and to also produce empty, non-infectious parvovirus capsids in order to deliver genetic information (WO 90/05538, May 31, 1990). Construction of recombinant parvoviral vectors is also described in Tattersall et al. U.S. Pat. No. 5,853,716.

Other delivery methods, vehicles or vectors are readily withing the skill of a practitioner in the art, and the foregoing should not be construed as limiting the delivery methods, vehicles or vectors contemplated as within the scope of the present invention.

E. Administration of Compounds of the Present Invention for Treatment of Tumors

The agents of the present invention can be administered via subcutaneous, intravenous, intramuscular, intraperitoneal, or buccal routes, direct injection into a tumor mass or in any manner that provides for entry of the nucleotide constructs of the invention into angiogenic epithelial cells. The dosage administered will be dependent upon the age, health, and weight of the recipient, kind of concurrent treatment, if any, frequency of treatment, and the nature of the effect desired.

In addition to the pharmacologically active agent, the compositions of the present invention may contain suitable pharmaceutically acceptable carriers comprising excipients and auxiliaries which facilitate processing of the active compounds into preparations which can be used pharmaceutically for delivery to the site of action. Suitable formulations for parenteral administration include aqueous solutions of the active compounds in water-soluble form, for example, water-soluble salts. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides. Aqueous injection suspensions may contain substances which increase the viscosity of the suspension include, for example, sodium carboxymethyl cellulose, sorbitol, and/or dextran. Optionally, the suspension may also contain stabilizers. Liposomes can also be used to encapsulate the agent for delivery into the cell.

In practicing the methods of this invention, the compounds of this invention may be used alone or in combination, or in combination with other therapeutic or diagnostic agents. In certain preferred embodiments, the compounds of this invention may be coadministered along with other compounds typically prescribed for these conditions according to generally accepted medical practice, including anti-angiogenic agents, such as angiostatin or endostatin expression vectors or proteins, or other anti-cancer therapeutics. As used herein, two agents are said to be administered in combination when the two agents are administered simultaneously or are administered independently in a fashion such that the agents will act at the same time. The compounds of this invention can be utilized in vivo, ordinarily in mammals, such as humans, sheep, horses, cattle, pigs, dogs, cats, rats and mice, or in vitro.

Therapeutically effective dosages may be determined by either in vitro or in vivo methods. For each particular compound of the present invention, individual determinations may be made to determine the optimal dosage required. The range of therapeutically effective dosages will be influenced by the route of administration, the therapeutic objectives and the condition of the patient, as well, for example, by the nature, stage and size of a tumor. For injection by hypodermic needle, it may be assumed the dosage is delivered into the body's fluids. For other routes of administration, the absorption efficiency must be individually determined for each compound by methods well known in pharmacology. Accordingly, it may be necessary for the therapist to titer the dosage and modify the route of administration as required to obtain the optimal therapeutic effect. The determination of effective dosage levels, that is, the dosage levels necessary to achieve the desired result, will be readily determined by one skilled in the art. Typically, applications of compound are commenced at lower dosage levels, with dosage levels being increased until the desired effect is achieved.

While individual needs vary, determination of optimal ranges of effective amounts of each component is within the skill of the art. The compounds of the invention can be administered intravenously or parenterally in an effective amount within the dosage range of about 0.01 mg to about 50 milligram/kg, preferably about 0.05 mg to about 5 mg/kg and more preferably about 0.2 mg to about 1.5 mg/kg on a regimen in a single or 2 to 4 divided daily doses and/or continuous infusion.

The nucleotide constructs of the invention may be administered in the form of liposome delivery systems, such as small unilamellar vesicles, large unilamellar vesicles and multilamellar vesicles. In particular, U.S. Pat. Nos. 5,837,283 and 5,948,767, EP Patent 921,193 to Mixson and Xu et al. (1997) Human Gene Therapy 8: 177-185 provide guidance as to the formulation of nucleotide construct/liposome complexes for intravenous or intraperitoneal use. Liposomes can be formed from a variety of lipids, such as cholesterol, stearylamine or phosphatidylcholines.

The compounds and constructs of the present invention may also be delivered by the use of antibodies, antibody fragments, growth factors, hormones, or other targeting moieties, to which the compound molecules are coupled. The compounds of this invention may also be coupled with suitable polymers as targetable drug carriers. Such polymers can include polyvinylpyrrolidinone, pyran copolymer, polyhydroxy-propylmethacrylamide-phenol, polyhydroxyethyl-aspartamide-phenol, or polyethyleneoxidepolylysine substituted with palmitoyl residues. Furthermore, compounds of the invention may be coupled to a class of biodegradable polymers useful in achieving controlled release of a drug, for example polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and cross linked or amphipathic block copolymers of hydrogels. Polymers and semipermeable polymer matrices may be formed into shaped articles, such as stents, tubing, and the like.

F. Applicability to Various Angiogenesis-Associated Diseases

There are several neoplastic and non-neoplastic diseases associated with proliferating or angiogenic epithelial cells. As discussed in Davis-Smyth et al., U.S. Pat. No. 5,952,199, these diseases include solid and metastatic tumors, and diseases such as rheumatoid arthritis, psoriasis, atherosclerosis; diabetic retinopathy, retrolenta fibroplasia, neovascular glaucoma, age-related macular degeneration, hemangiomas, immune rejection of transplanted corneal or other tissue, and chronic inflammation.

Conventional therapies for these disease are varied. For example, cancers may be treated by a wide variety of chemotherapeutics. Rheumatoid arthritis is often treated with aspirin or aspirin substitutes such as ibuprofen, corticosteroids or immunosuppressive therapy. Merck Manual (1992) 16th ed., pp. 1305-12. Atherosclerosis treatment is directed towards symptomatic conditions or risk factors, such as reducing circulating cholesterol levels or angioplasty. Merck Manual (1992) 16th ed., pp. 409-412. Diabetes mellitus can induce a range of condition, including diabetic atherosclerosis and diabetic retinopathy, which can be treated by controlling the primary diabetes or associated conditions-such as blood pressure. Merck Manual (1992) 16th ed., pp. 412-413, 1106-1125, 2383-2385. Psoriasis is most commonly treated with topical ointments and steroid treatments. Merck Manual (1992) 16th ed., pp. 2435-2437. Retrolenta fibroplasia is best treated by preventative oxygen and vitamin E treatments, although cryotherapeutic ablation may also be required. Merck Manual (1992) 16th ed., pp. 1975-1976. From the foregoing, it is clear that these angiogenesis-associated diseases do not share common treatment indications despite their shared angiogenic association.

Inhibition or prevention of angiogenesis provides a new and more global mechanism of treating such angiogenesis-associated diseases. In the Davis-Smyth et al. Patent, treatment is mediated by chimeric VEGF receptors which bind to and inactivate endogenous angiogenic VEGF protein with consequent decreases in the proliferation and angiogenesis of the vascular epithelium. In the present invention, the use of a nucleotide construct according to the present invention in an angiogenic or proliferating endothelial cell results in cell death, effectively terminating or preventing the angiogenic process associated with the foregoing diseases relatively early in the course of the disease.

G. Combination or Co-Administration Therapies

As contemplated, the present invention also relates to the combination or co-administration of the compounds disclosed herein by the associated inventive methods, together with the administration of other therapies, angiogenesis inhibitors and/or other anti-tumor agents. Such other therapies, angiogenesis inhibitors and agents are well known, for example, to ophthalmologists and oncologists. Such other agents and associated methods to be used in combination with the constructs and methods of the present invention include conventional chemotherapeutic agents, radiation therapy, immunomodulatory agents, gene therapy, and the use of various other compositions such as immunotoxins and anti-angiogenic formulations, such as angiostatin or endostatin, as are disclosed, for example, in U.S. Pat. No. 5,874,081 to Parish et al. (1999) and U.S. Pat. No. 5,863,538 to Thorpe et al. (1999) or are otherwise known in the art. Combination or co-administration therapies based on the present invention and the conventional therapies for angiogenesis associated diseases, such as discussed in Section F above, are also particularly contemplated.

In light of the foregoing general discussion, the specific examples presented below are illustrative only and are not intended to limit the scope of the invention. Other generic and specific configurations will be apparent to those persons skilled in the art.

### EXAMPLES

### Example 1

### Transfection of HUVEC Using SV40 Promoter Plus Diphtheria Toxin Construct

Materials and Methods. DNA sequences encoding the diphtheria toxin A chain (“DT-A”) or attenuated mutant diphtheria toxin A chain (“DT-A-tox176) were cloned downstream of the SV40 early promoter, creating plasmids pSV2A-DT-A (Robinson and Maxwell, Human Gene Ther. (1995) 6:127-143) and its tox176 derivative. Human umbilical vein endothelial cells (HUVEC) and growth medium were purchased from Cascade Biologicals, Inc., and the cells were cultured according to the instructions provided by this manufacturer.

Transfection and Luciferase Assays. Efficient transient transfection was achieved using the electroporation method Maxwell and Maxwell (1988) DNA 7:557-562. Transient cotransfection assays used the double reporter (pG4RLUC plus pSG236) system described in Maxwell et al., 1992. Luciferase assays used a kit from Promega, Inc., with a luminometer from Turner Instruments, Inc.

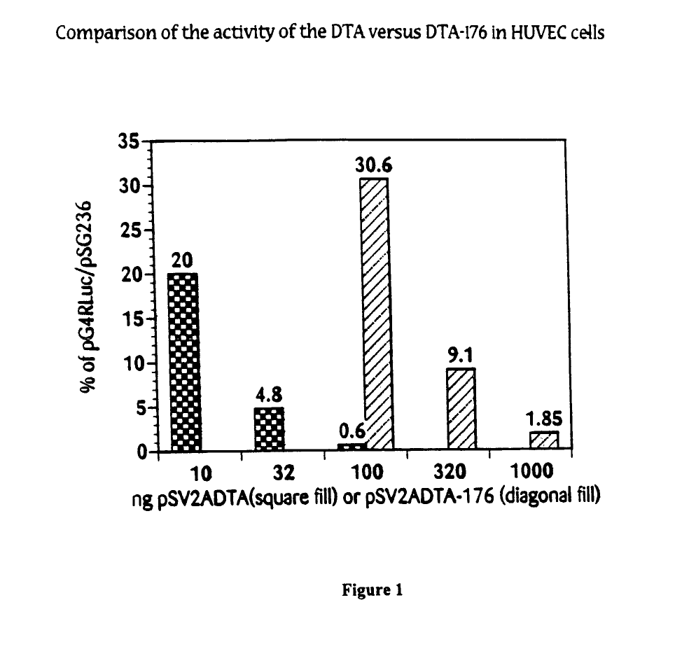

HUVEC were cotransfected luciferase reporter plasmids and with increasing amounts of the DT-A or DT-A-tox176 expressing plasmids. The results, shown in FIG. 1, confirmed that HUVEC are highly susceptible to the toxic effect of DT-A expression, which is mediated by rapid inhibition of protein synthesis. As shown in FIG. 1, transfection with increasing amounts of DT-A plasmid caused a progressive decrease in luciferase activity in the transient cotransfection assay in HUVEC (Maxwell et al., 1986). The cells were also susceptible to expression of the attenuated mutant, tox176, although approximately 30 fold higher levels were required for comparable inhibition, consistent with previous findings (Maxwell et al., 1987).

### Example 2

### Transfection of HUVEC using E-selectin Promoter Plus Diphtheria Toxin

Materials. The E-selectin promoter (nucleotides −523 to +33; Whitley et al., 1994) was amplified by PCR from human genomic DNA and was cloned into a derivative of pTHA7 (Maxwell et al., 1989) to generate pE-S-DT-A. The sequence of the promoter was confirmed by DNA sequencing to exclude possible PCR errors. Plasmid pE-S-GFP-LUC was constructed by substituting the GFP-Luciferase reporter gene (Day et al., 1998) for the DT-A sequence in pE-S-DT-A. TNFα was purchased from R&D Systems, Inc.

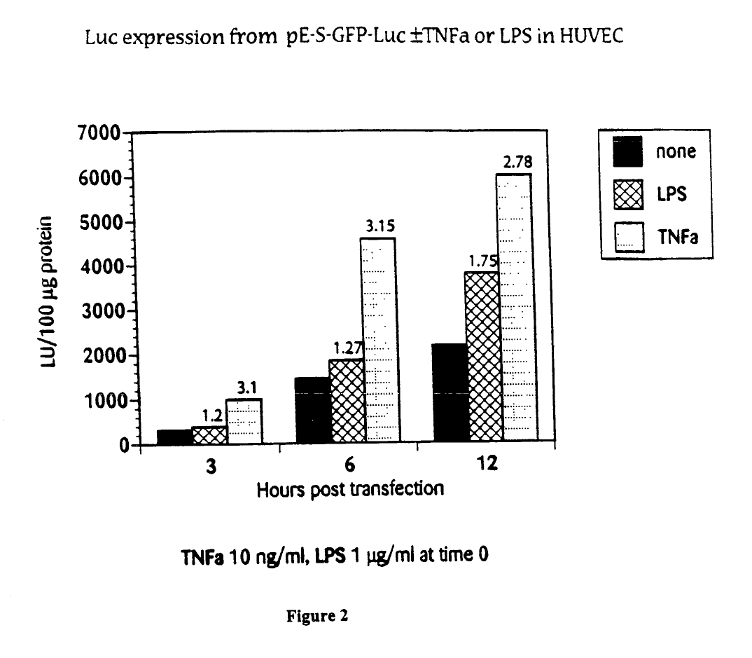

Plasmid pE-S-GFP-Luc (0.5 to 5 μg of DNA) was transfected into HUVEC and luciferase activity was measured at 3, 6 and 12 hours post transfection. Parallel transfections were conducted in the presence of 10 μg/ml TNFα or 1.0 μg/ml lipopolysaccharide (“LPS”). The results, shown in FIG. 2, demonstrated that the cloned E-selectin promoter is active in HUVEC as indicated by luciferase expression from the reporter plasmid pE-S-GFP-LUC. In addition, the results confirm the expected stimulation of this promoter (approximately 3 fold) in response to TNFα. Lipolysaccharide (LPS) also stimulated reporter expression, although to a lesser extent.

The effect of DT-A expression on HUVEC was assayed by cotransfecting LPS stimulated HUVEC with a luciferase expression vector PSV2A Luc and increasing amounts of p-E-S-DT-A. Lucerifase activity was measured at 9.5 and 19.5 hours post transfection.

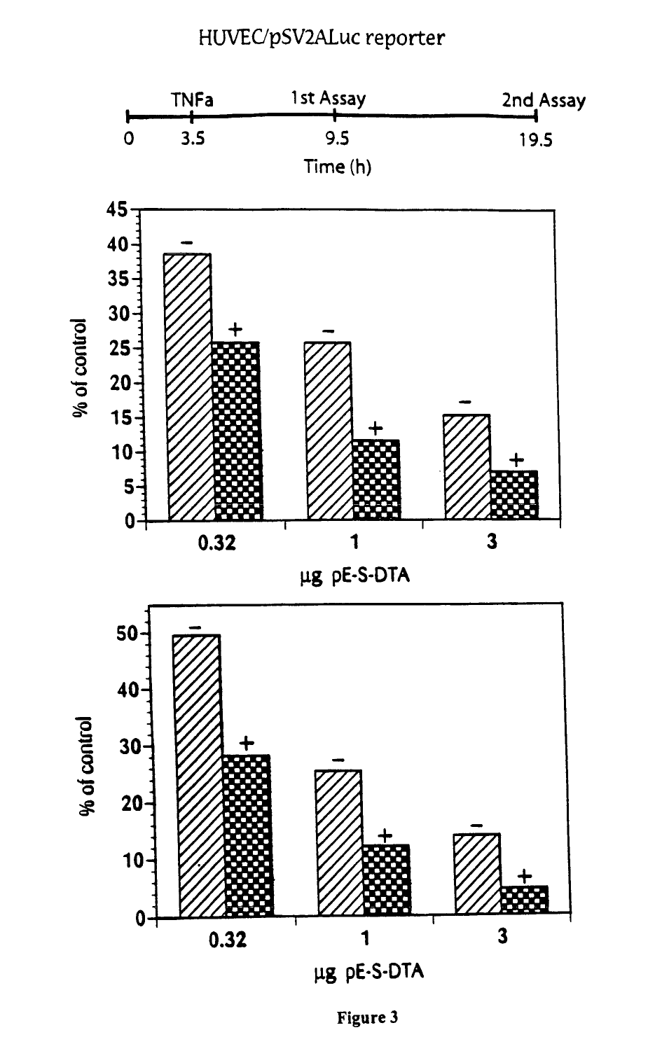

The results, shown in FIG. 3, demonstrate protein synthesis was inhibited by DT-A expression from the E-selectin promoter (plasmid pE-S-DT-A) in HUVEC, as well as that increased inhibition occurred in cells activated by TNFα. Both assay times gave similar results, including stimulation of DT-A expression by TNFα. These data indicate that the E-selectin promoter will be useful in targeting DT-A expression and toxicity to the activated endothelium of tumor vasculature in vivo.

### Example 3

### Transfection of HUVEC with Ets-1 Promoter/Diphtheria Toxin Constructs

Materials. Primary culture human umbilical vein endothelial cells (HUVEC) were obtained from Clonetics Corp. (San Diego, Calif.) and were grown in M199 medium supplemented with 20% FCS (Hy-Clone, Logan, Utah) 30 mg endothelial cell growth supplement (Collaborative Biomedical, Bedford, Mass.) and 25 mg Heparin in gelatin-coated tissue culture plates. Primary culture were passaged every 4-6 d and experiments were performed on 3-6 passages from primary culture.

Cell line 293 (ECACC Ref No: 8512062) is a human epithelial cell line cultured in EMEM Medium with 2 mM Glutamine, 1% non essential amino acids, 10% horse serum or 10% foetal bovine serum. Cell line BHK 21 (clone 13) (ECACC Ref No: 85011433) is a fibroblast cell line cultured in GMEM Medium supplemented with 2 mM Glutamine, 5% tryptose phosphate broth and 5-10% foetal bovine serum.

Cloning of the 5′-flanking region of the human ets-1 gene. Human genomic DNA was isolated from HUVEC cells with the Wizard Genomic DNA purification kit according to the supplier. A series of ETS-promoters was generated by PCR amplification and cloned into pGL3-Basic vector (Promega). The promoter inserts of all reporter constructs were confirmed by dideoxynucleotide sequencing. The promoters were also cloned into the Diphtheria A-chain coding promoterless plasmid pTHA7 (Maxwell et al.)

Transfection and Luciferase assay. HUVEC and control cell lines 293 and BHK 21 were transfected using calcium phosphate technique as previously described (Sambrook et al. 1989 Molecular cloning Cold Spring Harbor Laboratory, Cold Spring Harbor, N.Y.). Cells were transfected with 12 ug of reporter plasmid (E-Selectin-promoter-Luciferase, ets-1-promoter-Luciferase, control: SV40-promoter-Luciferase), and in the case of the ets-1 promoter plasmid, cotransfected with 2 μg of an ets-1 expression plasmid. After 19½ hours following transfection (with or without TNF-α or growth factor induction), samples were collected and assayed for Luciferase by using commercially available kits (Promega). Results show a strong expression of the E-selectin and ets-1 promoter constructs in HUVEC in comparison to the control cell lines. When these cell lines were cotransfected with luciferase reporter plasmid and ets-1 promoter/DT-A expression constructs, luciferase activity was greatly suppressed in transfected HUVEC relative to control cell lines.

### Example 4

### Lipofection with the Flt-1 Promoter/Diphtheria Toxin Constructs

The Flt-1 promoter as disclosed in Patent No. WO 97/17359 is cloned upstream of the Diphtheria A-chain coding promoterless plasmid pTHA7. The promoter inserts of the constructs are confirmed by dideoxynucleotide sequencing, as described in Example 3. These constructs are then formulated in cationic liposomes according to the McDonald et al. U.S. Pat. No. 5,837,283, for example DOTAP:CHOL 50:50. HUVEC, 293 and BHK 21 cells are prepared as described in Example 3. After lipofection of the construct into cells, 80% of the HUVEC cells are apoptotic as measured by the TUNEL assay (Gavrieli (1992) J. Cell Biol. 119:493-501). BHK 21 and 293 cells are less that 5% apoptotic under similar conditions.

### Example 5

### Preparation and Transfection of the KDR-Promoter/Diphtheria Toxin Constructs.

The KDR-promoter as disclosed in published PCT Patent Application No. WO 97/00957 is cloned into the Diphtheria A-chain coding promoterless plasmid pTHA7. HUVEC, 293 and BHK 21 cells are cultured as described in Example 3. The resulting plasmid is cotransfected into HUVEC and the non-endothelial control cells with a Luciferase reporter plasmid as described in Example 2. Luciferase activity is greatly suppressed in HUVEC relative to control cell-lines.

### Example 6

### Transfection Using the E-Selectin Promoter Linked to the VEGF Enhancer and the Luciferase Gene.

The VEGF enhancer from nucleotide −985 to −951 (relative to the transcription initiation site) of the VEGF promoter as disclosed in Liu et al. 1995 Circulation Research 77:638) is cloned upstream of the E-selectin promoter pE-S-GFP-LUC (see Example 2) and confirmed by dideoxy sequencing. This plasmid is then transfected in HUVEC, which were incubated under either hypoxic (0% O₂) or normoxic (21% O₂) conditions. Luciferase activity is induced at least 3-5-fold by hypoxia.

### Example 7

### Transfection with a Retrovirus Construct

Retroviral ets-1 promoter/DT-A particles are prepared as described in Danos et al. Cultured HUVEC and control cells are infected with these particles. At 24 hours post-infection, the HUVEC are about 80% apoptotic.

### Example 8

### Tumor Regression in Nude Mice.

The DT-A gene under the control of the E-selectin promoter is cloned into a plasmid and formulated into liposomes and injected into tumor-bearing nude test mice. Parallel injections into control tumor bearing nude mice are made with liposomes containing plasmids lacking the DT-A insert. After two injections, the test and control mice are sacrificed fourteen days post injection and examined by dissection. The test mice display statistically significant decreases in tumor mass.

### Example 9

### Treatment of Cancer Patients with Solid Tumors.

The E-selectin promotor is inserted upstream of the DT-A gene according to Example 2. This construct is packaged in a liposomal formulation as described in the McDonald et al. Patent. Therapeutically effective amounts of the formulation are administered intravenously to a patient suffering from one or more solid tumor growths. Therapy is maintained until tumor regression has occurred as determined by one or more markers of regression, including a decline in circulating tumor antigens and/or physical resorption. Subsequent continuous or periodic treatments with the formulation are optionally indicated as a prophylactic or as a means to ensure total tumor regression.

### Example 10

### Treatment of Patients with Retrolenta Fibroplasia.

A patient suffering from retrolenta fibroplasia is treated with cryotherapeutic ablation. A therapeutic DT-A formulation as described in Example 4 also is administered to the patient. Revascularization of the ablated area is reduced or prevented.

### Example 11

### Combination Therapy with Diphtheria Toxin.

A patient suffering from one or more solid tumors is treated according to Example 9. Following the initial course of DT-A therapy, the patient is subjected to traditional chemotherapy and/or radiation therapy. Therapeutic progress is monitored as described in Example 9. Use of the combination therapy permits reduced exposure of the patient to radiation or chemotherapeutics.

### Example 12

### Coadministration of a Diphtheria Toxin Construct with a Second Agent.

A liposomal formulation as described in Example 4 is co-formulated with an immunotoxin as described in Thorpe et al. U.S. Pat. No. 5,965,132. Therapeutically effective amounts of co-formulated liposomes are administered to a patient suffering from one or more solid tumors. Tumor regression is observed.

### Example 13

### Transfection Using the E-Selectin Promoter Linked to a Pentamer of an ETS-1 Binding Site and the Ludiferase Gene.

A pentamer of an ETS-1 binding site was cloned in front of the E-selectin promoter and the luciferase gene (etbz-ES). This plasmid was cotransfected with an ets-1 expression plasmid in 324K cells. Luciferase activity was induced 7 fold in comparison to an identical plasmid lacking the enhancer (E-S).

It should be understood that the foregoing discussion and examples merely present a detailed description of certain preferred embodiments. It therefore should be apparent to those of ordinary skill in the art that various modifications and equivalents can be made without departing from the spirit and scope of the invention. For example, it is contemplated that other highly toxic proteins and peptides could be encoded by the isolated nucleic acids and pharmaceutical compositions of the present invention. All journal articles, other references, patents and patent applications that are identified in this patent application are incorporated by reference in their entirety.

1. An isolated nucleic acid molecule comprising a sequence that encodes a highly toxic protein and regulatory elements effective to selectively express the sequence in angiogenic endothelial cells.

2. The isolated nucleic acid of claim 1, wherein the protein is selected from the group consisting of diphtheria toxin, pseudomonas exotoxin, cholera toxin, shiga-like toxin I, ricin A, trichoanguin, alpha-trichosanthin, abrin A, modeccin, Granulysin and related toxins, and highly toxic mutants and fragments of the foregoing.

3. The isolated nucleic acid of claim 2 wherein the protein is diphtheria toxin or diphtheria toxin mutants and fragments thereof.

4. The isolated nucleic acid of claim 1, further comprising a promoter that is selectively active in angiogenic endothelial cells.

5. The isolated nucleic acid of claim 4, wherein the promoter is selected from the group consisting of E-selectin promoter, ets-1 promoter, endosialin promoter, flt-1 promoter, flk-1 promoter and KDR promoter.

6. The isolated nucleic acid of claim 4, further comprising an enhancer, in one or more copies, that is active in endothelial cells.

7. The isolated nucleic acid of claim 6, further comprising an enhancer, in one or more copies, that is selectively active in angiogenic endothelial cells.

8. The isolated nucleic acid of any of claims 1-7, wherein the enhancer is selected from the group consisting of the HB-EGF enhancer, an enhancer from the first intron of the mouse tie2 gene, an enhancer from the first intron of the VEGF receptor (flk-1/KDR) gene, an enhancer from the first intron of the ets-1 gene, and a hypoxic response enhancer element that is selectively active under hypoxic conditions.

9. The isolated nucleic acid of claim 8, wherein the hypoxic response enhancer element is selected from the group consisting of the erythropoietin HRE element, pyruvate kinase HRE element, enolase HRE element, endothelin-1 HRE element and the VEGF hypoxic regulated enhancer.

10. The isolated nucleic acid of claim 1, further comprising a nucleotide sequence corresponding to an ETS binding site in one or more copies.

11. A delivery vehicle comprising the isolated nucleic acid of any of claims 1 to 7 or 10.

12. The delivery vehicle of claim 11, further comprising a component selected from the group consisting of retroviral particles, parvoviral particles, liposomes, cationic liposomes, liposomes or cationic liposomes with attached agents that target the liposomes selectively to endothelial and angiogenic endothelial cells respectively, and polynucleotide lipid complexes.

13. A pharmaceutical composition comprising the isolated nucleic acid of any of claims 1-7 or 10.

14. The pharmaceutical composition according to claim 13, further comprising a component selected from the group consisting of retroviral particles, parvoviral particles, liposomes, cationic liposomes, liposomes or cationic liposomes with attached agents that target the liposomes selectively to endothelial and angiogenic endothelial cells respectively, and polynucleotide lipid complexes.

15. The pharmaceutical composition of claim 13, in combination with a written label or package insert indicating that the composition may be used to treat an angiogenesis associated disease.

16. A method of treating an angiogenesis associated disease in a patient, comprising the administration to the patient of a therapeutically effective amount of a pharmaceutical composition according to claim 13.

17. The method of treating an angiogenesis associated disease according to claim 16, wherein the pharmaceutical composition further comprises a component selected from the group consisting of retroviral particles, parvoviral particles, liposomes, cationic liposomes and polynucleotide lipid complexes.

18. The method of claim 16, wherein the angiogenesis associated disease is selected from the group consisting of rheumatoid arthritis, atherosclerosis, diabetes mellitus, retinopathy, psoriasis and retrolental fibroplasia.

19. The method of claim 16, wherein the angiogenesis associated disease is cancer, and the administration of the pharmaceutical composition results in tumor regression as indicated by one or more measures of tumor regression.

20. A method of treating pathological blood vessel proliferation in a patient, comprising the administration to the patient of a therapeutically effective amount of a pharmaceutical composition according to claim 13.

21. The method of claim 20, wherein the pathological blood vessel proliferation is symptom of a disease selected from the group consisting of rheumatoid arthritis, atherosclerosis, diabetes mellitus, retinopathy, psoriasis and retrolental fibroplasia.

22. The method of claim 21, wherein the pharmaceutical composition is administered in combination with radiation therapy, chemotherapy, an anti-angiogenic agent or immunomodulatory agent.

23. The pharmaceutical composition of claims 13 or 14, further comprising at least one additional therapeutic agent selected from the group consisting of chemotherapeutics, immunomodulatory agents, angiogenesis inhibitors and mixtures thereof.
